(19) United States
(12) Patent Application Publication
(10) Pub. No.: US 2014/0182585 A1
(43) Pub. Date: Jul. 3, 2014

(54) AEROSOL CONTAINER FOR FOAMABLE COMPOSITIONS

(71) Applicant: Foamix Ltd., Rehovot (IL)

(72) Inventors: Dov Tamarkin, Macabim (IL); Doron Friedman, Karmei Yosef (IL); Meir Eini, Ness Ziona (IL)

(73) Assignee: Foamix Ltd., Rehovot (IL)

(21) Appl. No.: 14/200,126

(22) Filed: Mar. 7, 2014

Related U.S. Application Data
(63) Continuation of application No. 11/041,921, filed on Jan. 24, 2005.
Application No. 11/041,921 is a continuation-in-part of application No. PCT/IB03/05527, filed on Oct. 24, 2003.
Application No. 11/041,921 is a continuation-in-part of application No. 10/911,367, filed on Aug. 4, 2004.
(60) Provisional application No. 60/429,546, filed on Nov. 29, 2002.
(60) Provisional application No. 60/492,385, filed on Aug. 4, 2003.

(30) Foreign Application Priority Data
Oct. 25, 2002 (IL) 152486

(51) Int. Cl. A61M 11/04; A61K 31/41

(52) U.S. Cl. CPC A61M 11/04; A61K 31/41
USPC 128/200.23

(57) ABSTRACT

A composition and therapeutic kit provide a therapeutic azole with increased solubility. The kit includes an aerosol packaging assembly containing a container accommodating a pressurized product and an outlet capable of releasing the pressurized product as a foam. The pressurized product includes a foamable composition including:
i. a therapeutic azole, wherein the solubility of the azole in the composition before foaming is less than the solubility of the azole in the composition after foaming;
ii. at least one organic carrier selected from the group consisting of a hydrophobic organic carrier, a co-solvent, an emollient and mixtures thereof, at a concentration of about 2% to about 50% by weight;
iii. a surface-active agent;
iv. about 0.01% to about 5% by weight of at least one polymeric additive selected from the group consisting of a bioadhesive agent, a gelling agent, a film forming agent and a phase change agent;
v. water; and
vi. liquefied or compressed gas propellant at a concentration of about 3% to about 25% by weight of the total composition.

### CROSS REFERENCE TO RELATED APPLICATIONS

This application is a continuation of U.S. application Ser. No. 11/041,921, filed on Jan. 24, 2005, which is a continuation-in-part application of co-pending International Patent Application No. IB03/005527, designating the United States and filed on Oct. 24, 2003, which claims the benefit of priority under 35 U.S.C. §119(e) to U.S. Patent Application Ser. No. 60/429,546, filed on Nov. 29, 2002, both entitled “Cosmetic and Pharmaceutical Foam,” and which claims the benefit of priority under 35 USC§119(a) to Israeli Patent Appl. No. 152486, filed Oct. 25, 2002, all of which are hereby incorporated in their entirety by reference.

U.S. application Ser. No. 11/041,921, filed on Jan. 24, 2005 is a continuation-in-part application of co-pending U.S. patent application Ser. No. 10/911,367, filed on Aug. 4, 2004, which claims the benefit of priority under 35 U.S.C. §119(e) to U.S. Patent Application Ser. No. 60/492,385, filed on Aug. 4, 2003, both entitled “Foam Carrier Containing Amphiphilic Copolymer Gelling Agent” all of which are hereby incorporated in their entirety by reference.

### BACKGROUND OF THE INVENTION

Certain active agents, present difficult problems in formulating such active agents for effective administration to patients due to their poor solubility in their designated carrier. A well-designed therapeutic product must, at a minimum, be capable of presenting a therapeutically effective amount of the therapeutic agent to the desired absorption site, in a solubilized form. Such agents penetrate better into their target site and thus, are expected to exert their therapeutic benefit in an improved fashion.

Therapeutic azoles contain an azole ring structure with a wide variety of complex side-chains. Imidazole antifungal agents inhibit the synthesis of ergosterol by blocking the action of 14-alpha-demethylase.

Metronidazole, 1-(2-hydroxyethyl)-2-methyl-5-nitroimidazole, is a therapeutic azole, which has long been known as an effective drug to treat a variety of disorders, and is especially well known for the treatment of various protozoal diseases. As a topical therapy, Metronidazole has also been shown to be useful in treating various skin disorders, including acne rosacea, bacterial ulcers, and perioral dermatitis. Metronidazole has been found to have an anti-inflammatory activity when used topically to treat dermatologic disorders.

Compositions containing therapeutic azoles for treatment of dermatologic and gynecological disorders are available in cream, lotion and gel forms.

For example, commercially available Metronidazole gel, cream and lotion products, namely Metrogel®, Metrocream® and Metrolotion® (Galderma Laboratories, Inc.), contain 0.75% Metronidazole, and are applied twice daily to affected areas. Metrogel-vaginal® (3M), also containing 0.75% Metronidazole, is available for intravaginal administration for bacterial vaginosis. One commercially available Metronidazole cream product, Noritate® (Dermik Laboratories, Inc.) contains 1% Metronidazole and is directed to be applied once daily to affected areas. Since the soluble concentration of Metronidazole is 0.75%, in higher concentration Metrnidazole is expected to be in suspension, as indeed, is the case in Noritate® products.

For the treatment of many dermatological and mucosal disorders, it is preferable to use a formulation wherein the drug is fully dissolved, rather than in suspension, in order to attain optimal bioavailability of the drug in its target site. However, products, as listed above are limited to a concentration of Metronidazole of 0.75% because of the poor solubility of Metronidazole in water. Metronidazole is practically insoluble in oils.

U.S. Pat. No. 4,837,378 describes topical aqueous single-phase compositions containing 0.25 to 1.0 wt % Metronidazole. U.S. Pat. No. 6,468,989 discloses an aqueous solution of Metronidazole in which the concentration of Metronidazole is higher than 0.75%. The solution contains the solubility enhancer hydroxypropyl-betacyclodextrin and may additionally contain niacinamide.

U.S. Pat. No. 5,840,744 discloses a non-flowing composition containing low doses of Metronidazole in a composition that includes a buffer system maintaining the composition at a pH value in the range of about 3.75 to about 4.25. The viscosity of the composition is at least sufficient to maintain a product composition of this invention in a non-flowing state.

WO 2004/112780 teaches a tinted topical pharmaceutical composition containing Metronidazole and at least one dye.

U.S. Pat. No. 6,383,471 discloses a pharmaceutical composition including a hydrophobic therapeutic agent having at least one ionizable functional group, and a carrier. The carrier includes an ionizing agent capable of ionizing the functional group, a surfactant, and optionally solubilizers, triglycerides, and neutralizing agents. The compositions of the invention are particularly suitable for use in oral dosage forms.

### SUMMARY OF THE INVENTION

The present invention provides a therapeutic kit including a therapeutic azole with increased solubility. The kit includes an aerosol packaging assembly having a container accommodating a pressurized product and an outlet capable of releasing the pressurized product as a foam. The pressurized product is a foamable composition including:

i. a therapeutic azole, wherein the solubility of the azole in the composition before foaming is less than the solubility of the azole in the composition after foaming;

ii. at least one organic carrier selected from the group consisting of a hydrophobic organic carrier, a co-solvent, an emollient and mixtures thereof, at a concentration of about 2% to about 50%;

iii. a surface-active agent;

iv. about 0.01% to about 5% of at least one polymeric additive selected from the group consisting of a bioadhesive agent, a gelling agent, a film forming agent and a phase change agent;

v. water; and

vi. liquefied or compressed gas propellant at a concentration of about 3% to about 25% of the total composition.

In one or more embodiments, the foamable composition is an emulsion, e.g., an oil-in-water emulsion.

In one or more embodiments, at least a portion of the therapeutic azole is suspended in the foamable composition.

In one or more embodiments, the outlet is a valve. The valve includes a stem with 1 to 4 apertures formed in the stem, wherein each aperture formed in the stem has a diameter of about 0.2 mm to about 1 mm.

In one or more embodiments, the foamable pharmaceutical composition is substantially alcohol-free.

In one or more embodiments, the foamable pharmaceutical composition further includes a foam adjuvant.

In exemplary embodiments, the therapeutic azole is an imidazole or triazole selected from the group of Miconazole, Ketoconazole, Clotrimazole, Econazole, Mebendazole, Bifonazole, Butoconazole, Fenticonazole, Isoconazole, Oxiconazole, Sertaconazole, Sulconazole, Thiabendazole, Tiaconazole, Fluconazole, Itraconazole, Ravuconazole and Posaconazole, Ribavirin, and salts and derivatives thereof. Additionally, the therapeutic azole can be a nucleoside or a nucleotide, or a nucleoside or nucleotide analogue, for example, selected from the group consisting of Acyclovir, Famciclovir, Gancyclovir, Valganciclovir and Abacavir.

In one or more embodiments, the foamable composition further includes at least one additional therapeutic agent is selected from the group consisting of an anti-infective, an antibiotic, an antibacterial agent, an antifungal agent, an antiviral agent, an antiparasitic agent, an antiinflammatory agent, an immunosuppressive agent, an immunomodulator, an immunoregulating agent, a hormonal agent, vitamin A, a vitamin A derivative, vitamin B, a vitamin B derivative, vitamin C, a vitamin C derivative, vitamin D, a vitamin D derivative, vitamin E, a vitamin E derivative, vitamin F, a vitamin F derivative, vitamin K, a vitamin K derivative, a wound healing agent, a disinfectant, an anesthetic, an analgesic, an antiallergic agent, a corticosteroid, a non-steroidal anti-inflammatory drug, an alpha hydroxyl acid, a beta-hydroxy acid, a neuropeptide, an allergen, an immunogenic substance, a haptene, an oxidizing agent, an antioxidant, a retinoid, an antiproliferative agent, an anticancer agent, a photodynamic therapy agent, an anti-wrinkle agent, a radical scavenger, a self-tanning agent, a skin whitening agent, a skin protective agent, an anti-cellulite agent, a massaging oil and an anti-wart agent, a refatting agent, a lubricating agent and mixtures thereof.

In another aspect of the present invention a pharmaceutical composition is provided, including:

a) Metronidazole, in a concentration of at least 1%;

b) at least one organic carrier selected from a hydrophobic organic carrier, a co-solvent, an emollient and mixtures thereof, at a concentration of about 2% to about 50% by weight;

c) a surface-active agent; and

d) about 0.01% to about 5% by weight of at least one polymeric agent selected from the group consisting of a bioadhesive agent, a gelling agent, a film forming agent and a phase change agent; preferably, wherein the Metronidazole is substantially dissolved in the composition.

The present invention further provides a method for enhancing the dermal or transdermal delivery of a therapeutic azole by releasing a foamed product from an aerosol packaging assembly including pressurized container and an outlet. The assembly houses a foamable composition including:

(i) a therapeutic azole;

(ii) at least one organic carrier selected from a hydrophobic organic carrier, a co-solvent, an emollient and mixtures thereof, at a concentration of about 2% to about 50% by weight;

(iii) about 0.1% to about 5% by weight of a surface-active agent;

(iv) about 0.01% to about 5% by weight of a polymeric additive selected from a bioadhesive agent, a gelling agent, a film forming agent and a phase change agent; and

(v) a liquefied or compressed gas propellant at a concentration of about 3% to about 25% by weight of the total composition. The solubility of the azole in the composition after foaming is greater than the solubility of the azole in the aerosol assembly, so that the foamed product delivers an azole of enhanced solubility to a dermal surface.

A further aspect of the present invention provides a method of producing a therapeutic kit including a therapeutic azole by providing a foamable composition including:

(i) a therapeutic azole at a therapeutically effective concentration;

(ii) at least one organic carrier selected from a hydrophobic organic carrier, a co-solvent, an emollient and mixtures thereof, at a concentration of about 2% to about 50% by weight;

(iii) a surface-active agent; and

(iv) about 0.01% to about 5% by weight of a polymeric additive selected from a bioadhesive agent, a gelling agent, a film forming agent and a phase change agent; introducing the foamable composition in an aerosol packaging assembly including a container suitable for containing a pressurized product and a valve capable of extruding a foam; and adding to the aerosol packaging assembly a liquefied or compressed gas propellant at a concentration of about 3% to about 25% by weight of the total composition.

### BRIEF DESCRIPTION OF THE DRAWING

The invention is described with reference to the figures which are presented for the purpose of illustration and are not intended to be limiting of the invention.

FIG. 1 is a schematic illustration of an aerosol valve suitable for use in the aerosol packaging assembly according to in one or more embodiments of the invention.

FIG. 2 present photographs of compositions comparing the microscopic evaluation of crystals in 1% Metronidazole compositions of (a) an emulsion and (b) a foamed composition according to one or more embodiments of the present invention and (c) the commercial 1% Metronidazole topical product—Noritate (Dermik Laboratories Ltd.)

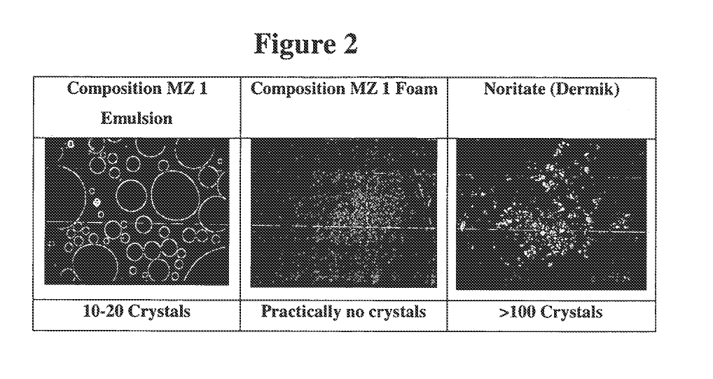

### DETAILED DESCRIPTION OF THE INVENTION

The present invention provides a therapeutic kit including a therapeutic azole with increased solubility. The increased solubility of the therapeutic azole provides a higher concentration of solubilized azole at the treatment site, which in turn results in its greater penetration into the target site. An enhanced therapeutic effect is observed.

In one aspect, the therapeutic kit includes an aerosol packaging assembly containing a pharmaceutical composition including a therapeutic azole in a concentration higher than its expected solubility concentration in the composition, as derived from the known solubility of said azole in the primary composition components; i.e., water and/or oil, as applicable. The threshold concentration of the azole in the pharmaceutical composition is elevated by at least 0.1 wt %, or at least 0.2 wt %.

In other aspects, the azole is suspended in the composition, and is released from the aerosol assembly as a foam. The therapeutic azole in the foamed product is more soluble than the azole in the foamable composition prior its to release from the aerosol assembly. An increase in solubility of at least 0.1 wt % or at least 0.2 wt % is observed.

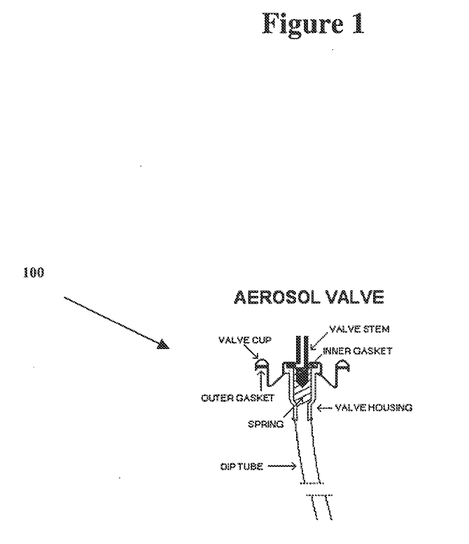

The aerosol packaging assembly typically includes a container suitable for accommodating a pressurized product and an outlet capable of releasing a foam. The outlet is typically a valve. FIG. 1 illustrates a typical aerosol valve 100. The valve is made up of the valve cup 110 typically constructed from tinplated steel, or aluminum, an outer gasket 120, which is the seal between the valve cup and the aerosol can (not shown), a valve housing 130, which contains the valve stem 132, spring 134 and inner gasket 136, and a dip tube 140, which allows the liquid to enter valve. The valve stem is the tap through which the product flows. The inner gasket 136 covers the aperture 150 (hole) in the valve stem. The valve spring 134 is usually made of stainless steel.

The valve stem is fitted with small apertures 150 (also termed “orifices” and “holes”), through which the product flows. Valves may contain one, two, three, four or more apertures, depending on the nature of the product to be dispensed. In the closed position, the aperture(s) is covered by the inner gasket. When the actuator is depressed it pushes the valve stem through the inner gasket, and the aperture(s) is uncovered, allowing liquid to pass through the valve and into the actuator.

The valve can have a stem with 1 to 4 apertures, or 1 to 2 apertures. Each aperture can have a diameter of about 0.2 mm to about 1 mm, or a diameter of about 0.3 mm to about 0.8 mm. The total aperture area, i.e., the sum of areas of all apertures in a given stem, is between about 0.01 mm² and 1 mm² or the total aperture area is between about 0.04 mm² and 0.5 mm².

### Pharmaceutical Composition

All % values are provided on a weight (w/w) basis.

According to one or more embodiments of the present invention, the foamable therapeutic composition for administration to a body surface, a body cavity or mucosal surface includes:

(1) a therapeutic azole, wherein the solubility of the azole in the composition before foaming is less than the solubility of the azole in the foamed composition.

(2) at least one organic carrier selected from a hydrophobic organic carrier, a co-solvent, an emollient and mixtures thereof, at a concentration of about 2% to about 5%, or about 5% to about 10%; or about 10% to about 20%; or about 20% to about 50% by weight;

(3) about 0.1% to about 5% by weight of a surface-active agent;

(4) about 0.01% to about 5% by weight of at eat one polymeric agent selected from a bioadhesive agent, a gelling agent, a film forming agent and a phase change agent; and

(5) a liquefied or compressed gas propellant at a concentration of about 3% to about 25% by weight of the total composition.

Water and optional ingredients are added to complete the total mass to 100%. Upon release from an aerosol container, the foamable composition forms an expanded foam suitable for topical administration. The composition may further include a foam adjuvant at a concentration less than about 5%.

According to one or more embodiments, the foamable composition is substantially alcohol-free, i.e., free of short chain alcohols. Short chain alcohols, having up to 5 carbon atoms in their carbon chain skeleton, such as ethanol, propanol, isopropanol, butanol, iso-butanol, t-butanol and pentanol, are considered less desirable solvents or co-solvents due to their skin-irritating effect. Thus, the composition is substantially alcohol-free and includes less than about 5% final concentration of lower alcohols, preferably less than about 2%, more preferably less than about 1%.

In one or more embodiments, at least a portion of the therapeutic azole is suspended in the composition.

In one or more embodiments, the foam composition is formulated as an oil-in-water emulsion or oil-in-water microemulsion.

When the composition as described herein is released from the therapeutic kit, it affords the therapeutic azole with increased solubility. In typical compositions, the threshold concentration of the azole in the foamed composition is elevated by at least about 0.1 wt % or at least about 0.2 wt %.

In a further aspect of the present invention, a pharmaceutical composition is provided including (1) Metronidazole in concentration of between at least 1%; (2) at least one organic carrier selected from a hydrophobic organic carrier, a co-solvent, an emollient and mixtures thereof, at a concentration of about 5% to about 10%; or about 10% to about 20%; or about 20% to about 50%; (3) about 0.1% to about 5% of a surface-active agent; and (4) about 0.01% to about 5% by weight of at least one polymeric agent selected from a bioadhesive agent, a gelling agent, a film forming agent and a phase change agent. Water and optional ingredients are added to complete the total mass to 100%.

In one or more embodiments, the concentration of surface-active agent about 0.1% to about 5%, or from about 0.2% to about 2%. In one or more embodiments, the pharmaceutical composition is formulated as an oil-in-water emulsion or oil-in-water microemulsion.

In one or more embodiments, the Metronidazole composition is flowable at ambient temperature; i.e., is not in a “non-flowing” state. The flowability property is useful, for example, for a composition that is used as a lotion. This property is also important, when the composition is intended to be delivered as aerosol spray or foam because the composition has to flow through the valve of the aerosol packaging assembly.

In certain embodiments, the pH of a Metronidazole composition can be lower than 3. In other embodiments, the pH of the Metronidazole composition is above 4.5, which is preferable for skin therapy. Yet, in other embodiments, the pH of a Metronidazole composition is between 3 and 4.5, which is suitable for vaginal therapy.

Surprisingly, a 1-2 wt % Metronidazole composition according to one or more embodiments of the invention demonstrates improved solubility over aqueous Metronidazole solutions. When visually examined under magnification, e.g. 100×, only a few crystals are detected microscopically, indicating that a major portion of the active agent is dissolved in the composition. Furthermore, when a foamable composition including Metronidazole (and containing Metronidazole crystals as described above) is foamed, the number of Metronidazole crystals decreases significantly, as visually observed under magnification (e.g., 100×). No additional crystals are observed at higher magnifications, e.g., 400× and 1000×, indicating that the reduction in presentation of crystals cannot be explained simply by breakdown of the crystals into a larger number of smaller crystals. Without being bound by any particular mode of operation, it is believed that the reduced crystallinity of the foamed product imparts increased solubility to the Metronidazole composition.

It has also been unexpectedly discovered that a foamable oil in water emulsion composition including Metronidazole also exhibits increased solubility of the azole, in both the prefoamed and foamed states.

At least a portion of the azole can be suspended in the foamable composition. As used herein the term “at least a portion of the azole can be suspended” means that a measurable fraction of the azole is in a solid and typically crystalline state in a pharmaceutical composition. A significant fraction can be visually detected under magnification by counting more than 50 crystals in an area of 1 mm² at 100× magnification. In the case of Metronidazole, this term corresponds to more than 20 crystals in an area of 1 mm² at 100× magnification.

The corresponding term “suspended”, as used herein, means that a significant fraction of the azole included in a pharmaceutical composition includes is in a solid state, as detected microscopically by counting more than 20 or more than 50 crystals in an area of 1 mm² at 100× magnification.

The corresponding term “soluble” or “substantially soluble,” as used herein, means that a significant fraction of the azole included in a pharmaceutical composition includes is solubilized in the composition, as detected microscopically by counting less than 20 crystals in an area of 1 mm² at 100× magnification.

Therapeutic azoles are pharmaceutically active compounds that are unsaturated five member ring heterocyclic compound, wherein one, two or three members of the ring are nitrogen atoms, as exemplified in a non-limiting way and illustrated in the following schemes:

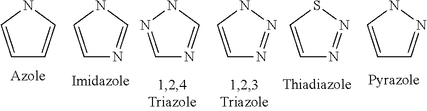

The therapeutic azole is a compound including an unsaturated five member ring heterocyclic compound, wherein one, two or three members of the ring are nitrogen atoms.

Examples of therapeutic imidazoles include, but are not limited to, Miconazole, Ketoconazole, Clotrimazole, Econazole, Mebendazole, Bifonazole, Butoconazole, Fenticonazole, Isoconazole, Oxiconazole, Sertaconazole, Sulconazole, Thiabendazole Tiaconazole. Such therapeutic imidazoles are mainly used as antifungal agents, yet several of them also possess other therapeutic benefits, such as anti-inflammatory, antibacterial and antiviral effects.

Therapeutic triazoles are exemplified by Fluconazole, Itraconazole, Ravuconazole and Posaconazole. Such therapeutic imidazoles are mainly used as antifungal agents, yet several of them also possess other therapeutic benefits, such as anti-inflammatory, antibacterial and antiviral effects.

Additional non-limiting exemplary classes of therapeutic azoles include, oxazoles, thiazoles, thiadiazoles and thiatriazoles, benzimidazoles, and salts and derivatives thereof.

Metronidazole, 1-(2-hydroxyethyl)-2-methyl-5-nitroimidazole, is a therapeutic azole, which has long been known as an effective drug to treat a variety of disorders, and is especially well known for the treatment of various protozoal diseases.

Several anti-viral agents, including, but not limited to, Acyclovir (also called acycloguanosine), Famciclovir, Gancyclovir, Valganciclovir, Abacavir, which are Nucleoside analogues, which include an imidazole moiety in them, and Ribavirin, which is a triazole. In one or more embodiments, the therapeutic azole is a nucleoside antibiotic or a nucleotide antibiotic.

A pharmaceutical composition or a foamable compositions according to one or more embodiments of the present invention may include one or more azole compounds, e.g., imidazoles, triazoles, nucleosides including imidazole moieties, or unsaturated five member ring heterocyclic compound, wherein one, two or three members of the ring are nitrogen atoms, at a concentration sufficient to leave at least a portion of the azole suspended in the composition.

In one or more embodiments, the therapeutic azole is Metronidazole, at a concentration of more than 0.75%, or between about 0.75% and about 5%, or, preferably between about 1% and about 2%.

In one or more embodiments, the therapeutic azole is Miconazole, at a concentration of more than 0.4%.

In one or more embodiments, the therapeutic azole is Miconazole, at a concentration between about 0.4% and about 4%.

In one or more embodiments, the therapeutic azole is Ketoconazole, at a concentration of more than 0.2%.

In one or more embodiments, the therapeutic azole is Ketoconazole, at a concentration between about 0.2% and about 4%.

In many cases, the inclusion of an additional therapeutic agent in the foamable pharmaceutical of the present invention, contributes to the clinical activity of the therapeutic azole. For example, it is known that keratolytic agents, such as alpha hydroxyl acids, beta hydroxyl acids, retinoids, etc., contribute to the clinical efficacy of an antifungal agent. Likewise, it is known, for example, that the addition of a second anti-infective agent, such as an antibacterial agent and antiviral agent, an anti-parasite agent or a second antifungal agent is beneficial in the treatment of a complex infectious condition. An additional non-limiting example is of an additional therapeutic agent is an anti-inflammatory agent, which contributes to therapy by treating the inflammatory reaction, which accompanies many infective conditions.

Thus, in one or more embodiments, the foamable composition further includes at least one additional therapeutic agent, in a therapeutically effective concentration.

In one or more embodiments, the at least one additional therapeutic agent is selected from the group consisting of an anti-infective, an antibiotic, an antibacterial agent, an antifungal agent, an antiviral agent, an antiparasitic agent, an antiinflammatory agent, an immunosuppressive agent, an immunomodulator, an immunoregulating agent, a hormonal agent, vitamin A, a vitamin A derivative, vitamin B, a vitamin B derivative, vitamin C, a vitamin C derivative, vitamin D, a vitamin D derivative, vitamin E, a vitamin E derivative, vitamin F, a vitamin F derivative, vitamin K, a vitamin K derivative, a wound healing agent, a disinfectant, an anesthetic, an analgesic, an antiallergic agent, a corticosteroid, a non-steroidal anti-inflammatory drug, an alpha hydroxyl acid, a beta-hydroxy acid, a protein, a peptide, a neuropeptide, a allergen, an immunogenic substance, a haptene, an oxidizing agent, an antioxidant, a retinoid, an antiproliferative agent, an anticancer agent, a photodynamic therapy agent, anti-wrinkle agent, a radical scavenger, a self-tanning agent, a skin whitening agent, a skin protective agent, an anti-cellulite agent, a massaging oil and an anti-wart agent, a refatting agent, a lubricating agent and mixtures thereof.

In one or more embodiments, the additional therapeutic agent is a peptide copper complex, i.e., a coordination compound comprising a peptide molecule and a copper(II) ion (i.e., Cu⁺²) non-covalently complexed therewith. The peptide molecule serves as the complexing agent by donating electrons to the copper ion to yield the non-covalent complex. The peptide molecule is a chain of two or more amino acid units or amino acid derivative units covalently bonded together via amide linkages.

According to one or more embodiments of the present invention, the additional active agent is a solid matter or particulate matter. Namely the composition includes at least one active agent that is substantially insoluble in the liquid carrier composition of the foamable composition. For definition purposes, solid matter shall include, but will not be limited to, material substantially insoluble in the foamable composition. By way of example, titanium dioxide, zinc oxide, zirconium oxide, iron oxide and mixtures thereof may be used as solid matter substances. In one embodiment the metal oxides are present in the amount of from about 0.1% to about 20%, or from about 0.5% to about 16%, or even from about 1% to about 10%, of the composition.

Generally, products for the prevention and treatment of fungal disorders, such as diaper dermatitis, would benefit from the combination of a metal or metalloid oxide protective agent and a suitable therapeutic azole.

Other suitable solid materials include silicon-containing solid matter such as silicon oxide, also termed “silica”, “fumed silica” and “silica gel”, a white or colorless insoluble solid (SiO2); and talc, which is fine grained mineral consisting of hydrated magnesium silicate; carbon, for example in the form of amorphous carbon or graphite; oxidizing agents, such as benzoyl peroxide, calcium and magnesium hypochlorite; metallic silver, in small particles, including nanocrystalline silver, which is used for antibacterial and wound healing purposes; other metal particles and mineral particles; cosmetic scrub materials, including, for example meals of strawberry seeds, raspberry seeds, apricot seeds, sweet almond, cranberry seeds; and pigments, which are insoluble in the composition of the present invention.

In certain cases, the disorder to be treated involves unaesthetic lesions that need to be masked. For example, rosacea involves papules and pustules, which can be treated with Metronidazole, as well as erythema, telangiectasia and redness, which do not respond to treatment with a therapeutic azole. Thus, in one or more embodiments, the additional active agent is a masking agent, i.e., a pigment. Non limiting examples of suitable pigments include brown, yellow or red iron oxide or hydroxides, chromium oxides or hydroxides, titanium oxides or hydroxides, zinc oxide, FD&C Blue No. 1 aluminum lake, FD&C Blue No. 2 aluminum lake and FD&C Yellow No. 6 aluminum lake.

The foamable composition of the present invention can be an emulsion, or microemulsion, including an aqueous phase and an organic carrier. The organic carrier is selected from a hydrophobic organic carrier (also termed herein “hydrophobic solvent”), an emollient, a co-solvent, and a mixture thereof.

A “hydrophobic organic carrier” as used herein refers to a material having solubility in distilled water at ambient temperature of less than about 1 gm per 100 mL, more preferable less than about 0.5 gm per 100 mL, and most preferably less than about 0.1 gm per 100 mL. It is liquid at ambient temperature. The identification of a hydrophobic organic carrier or “hydrophobic solvent”, as used herein, is not intended to characterize the solubilization capabilities of the solvent for any specific active agent or any other component of the foamable composition. Rather, such information is provided to aid in the identification of materials suitable for use as a hydrophobic carrier in the foamable compositions described herein.

In one or more embodiments, the hydrophobic organic carrier is an oil, such as mineral oil. Mineral oil (Chemical Abstracts Service Registry number 8012-95-1) is a mixture of aliphatic, naphthalenic, and aromatic liquid hydrocarbons that derive from petroleum. It is typically liquid; its viscosity is in the range of between about 35 CST and about 100 CST (at 40° C.), and its pour point (the lowest temperature at which an oil can be handled without excessive amounts of wax crystals forming so preventing flow) is below 0° C. The term hydrophobic does not include thick or semi-solid materials, such as white petrolatum, also termed “Vaseline”, is disadvantageous, due to its waxy nature and semi-solid texture. It is known to leave a waxy and sticky feeling after application and occasionally stain cloths. Thus, white petrolatum as well as other wax-like, semi-solid compounds are undesirable as a hydrophobic solvent according to the present invention.

According to one or more embodiments, hydrophobic solvents are liquid oils originating from vegetable, marine or animal sources. Suitable liquid oil includes saturated, unsaturated or polyunsaturated oils. By way of example, the unsaturated oil may be olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oils or mixtures thereof, in any proportion.

Suitable hydrophobic solvents also includes polyunsaturated oils containing omega-3 and omega-6 fatty acids. Examples of such polyunsaturated fatty acids are linoleic and linolenic acid, gamma-linoleic acid (GLA), eicosapentaenoic acid (EPA) and docosahexaenoic acid (DHA). Thus, the hydrophobic solvent can include at least 6% of an oil selected from omega-3 oil, omega-6 oil, and mixtures thereof.

Another class of hydrophobic solvents is the essential oils, which are considered “therapeutic oils”, which contain active biologically occurring molecules and, upon topical application, exert a therapeutic effect.

Another class of hydrophobic solvents includes liquid hydrophobic plant-derived oils, which are known to possess therapeutic benefits when applied topically.

Silicone oils also may be used and are desirable due to their known skin protective and occlusive properties. Suitable silicone oils include non-volatile silicones, such as polyalkyl siloxanes, polyaryl siloxanes, polyalkylaryl siloxanes and polyether siloxane copolymers, polydimethylsiloxanes (dimethicones) and poly(dimethylsiloxane)-(diphenyl-siloxane) copolymers. These are chosen from cyclic or linear polydimethylsiloxanes containing from about 3 to about 9, preferably from about 4 to about 5, silicon atoms. Volatile silicones such as cyclomethicones can also be used. Water-soluble silicones, such as dimethicone copolyol are not included in the definition of hydrophobic solvents.

In one or more embodiments, the solvent includes at least 2% by weight silicone oil or at least 5% by weight.

The solvent may be a mixture of two or more of the above hydrophobic solvents in any proportion.

A further class of solvents includes “emollients” that have a softening or soothing effect, especially when applied to body areas, such as the skin and mucosal surfaces. Emollients are not necessarily hydrophobic. Examples of suitable emollients include hexyleneglycol, propylene glycol, isostearic acid derivatives, isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, sucrose esters of fatty acids, octyl hydroxystearate and mixtures thereof. Other examples of other suitable emollients can also be found in the Cosmetic Bench Reference, pp. 1.19-1.22 (1996).

According to one or more embodiments of the present invention, the solvent includes a mixture of a hydrophobic solvent and an emollient. According to one or more embodiments, the foamable composition is a mixture of mineral oil and an emollient in a ratio between 2:8 and 8:2 on a weight basis.

A “co-solvent” is an organic solvent, typically soluble in both water and oil. Examples of co-solvents include polyols, such as glycerol (glycerin), propylene glycol, hexylene glycol, diethylene glycol, propylene glycol n-alkanols, terpenes, di-terpenes, tri-terpenes, terpen-ols, limonene, terpene-ol, 1-menthol, dioxolane, ethylene glycol, other glycols, sulfoxides, such as dimethylsulfoxide (DMSO), dimethylformanide, methyl dodecyl sulfoxide, dimethylacetamide, monooleate of ethoxylated glycerides (with 8 to 10 ethylene oxide units), azone (1-dodecylazacycloheptan-2-one), 2-(n-nonyl)-1,3-dioxolane, esters, such as isopropyl myristate/palmitate, ethyl acetate, butyl acetate, methyl proprionate, capric/caprylic triglycerides, octylmyristate, dodecyl-myristate; myristyl alcohol, lauryl alcohol, lauric acid, lauryl lactate ketones; amides, such as acetamide oleates such as triolein; various alkanoic acids such as caprylic acid; lactam compounds, such as azone; alkanols, such as dialkylamino acetates, and admixtures thereof.

According to one or more embodiments, the co-solvent is a polyethylene glycol (PEG) or PEG derivative that is liquid at ambient temperature, including PEG200 (MW (molecular weight) about 190-210 kD), PEG300 (MW about 285-315 kD), PEG400 (MW about 380-420 kD), PEG600 (MW about 570-630 kD) and higher MW PEGs such as PEG 4000, PEG 6000 and PEG 10000 and mixtures thereof.

The polymeric agent serves to stabilize the foam composition and to control drug residence in the target organ. Exemplary polymeric agents, are classified below in a non-limiting manner. In certain cases, a given polymer can belong to more than one of the classes provided below.

In one or more embodiments, the composition of the present invention includes at least one gelling agent. A gelling agent controls the residence of a therapeutic composition in the target site of treatment by increasing the viscosity of the composition, thereby limiting the rate of its clearance from the site. Many gelling agents are known in the art to possess mucoadhesive properties.

The gelling agent can be a natural gelling agent, a synthetic gelling agent and an inorganic gelling agent. Exemplary gelling agents that can be used in accordance with one or more embodiments of the present invention include, for example, naturally-occurring polymeric materials, such as locust bean gum, sodium alginate, sodium caseinate, egg albumin, gelatin agar, carrageenin gum, sodium alginate, xanthan gum, quince seed extract, tragacanth gum, guar gum, starch, chemically modified starches and the like, semi-synthetic polymeric materials such as cellulose ethers (e.g. hydroxyethyl cellulose, methyl cellulose, carboxymethyl cellulose, hydroxy propylmethyl cellulose), guar gum, hydroxypropyl guar gum, soluble starch, cationic celluloses, cationic guars, and the like, and synthetic polymeric materials, such as carboxyvinyl polymers, polyvinylpyrrolidone, polyvinyl alcohol, polyacrylic acid polymers, polymethacrylic acid polymers, polyvinyl acetate polymers, polyvinyl chloride polymers, polyvinylidene chloride polymers and the like. Mixtures of the above compounds are contemplated.

Further exemplary gelling agents include the acrylic acid/ethyl acrylate copolymers and the carboxyvinyl polymers sold, for example, by the B.F. Goodrich Company under the trademark of Carbopol® resins. These resins consist essentially of a colloidal water-soluble polyalkenyl polyether crosslinked polymer of acrylic acid crosslinked with from 0.75% to 2% of a crosslinking agent such as polyallyl sucrose or polyallyl pentaerythritol. Examples include Carbopol® 934, Carbopol® 940, Carbopol® 950, Carbopol® 980, Carbopol® 951 and Carbopol® 981. Carbopol® 934 is a water-soluble polymer of acrylic acid crosslinked with about 1% of a polyallyl ether of sucrose having an average of about 5.8 allyl groups for each sucrose molecule.

In one or more embodiment, the composition of the present invention includes at least one polymeric agent, which is a water-soluble cellulose ether. Preferably, the water-soluble cellulose ether is selected from the group consisting of methylcellulose, hydroxypropyl cellulose, hydroxypropyl methylcellulose (Methocel), hydroxyethyl cellulose, methylhydroxyethylcellulose, methylhydroxypropylcellulose, hydroxyethylcarboxymethylcellulose, carboxymethylcellulose and carboxymethylhydroxyethylcellulose. More preferably, the water-soluble cellulose ether is selected from the group consisting of methylcellulose, hydroxypropyl cellulose and hydroxypropyl methylcellulose (Methocel).

The use of a water soluble cellulose ether is especially advantageous when the therapeutic azole is Metronidazole, since such polymers surprisingly contribute to the dissolution of the therapeutic azole in a composition including both organic carrier and water. Without being bound by any particular mode of operation, it is believed that this effect results from interactions between the emulsion components (water, organic carrier and surface active agent) and the gelling agent, which contribute to the solubility of the therapeutic azole in the emulsion interface. In one or more embodiments, the composition includes a combination of a water-soluble cellulose ether; and a naturally-occurring polymeric materials, selected from the group including xanthan gum, guar gum, carrageenan gum, locust bean gum and tragacanth gum.

Yet, in other embodiments, the gelling agent includes inorganic gelling agents, such as silicone dioxide (fumed silica).

Mucoadhesive/bioadhesion has been defined as the attachment of synthetic or biological macromolecules to a biological tissue. Mucoadhesive agents are a class of polymeric biomaterials that exhibit the basic characteristic of a hydrogel, i.e. swell by absorbing water and interacting by means of adhesion with the mucous that covers epithelia.

Compositions of the present invention may contain a mucoadhesive macromolecule or polymer in an amount sufficient to confer bioadhesive properties. The bioadhesive macromolecule enhances the delivery of biologically active agents on or through the target surface. The mucoadhesive macromolecule may be selected from acidic synthetic polymers, preferably having at least one acidic group per four repeating or monomeric subunit moieties, such as poly(acrylic)- and/or poly(methacrylic) acid (e.g., Carbopol®, Carbomer®), poly(methylvinyl ether/maleic anhydride) copolymer, and their mixtures and copolymers; acidic synthetically modified natural polymers, such as carboxymethylcellulose (CMC); neutral synthetically modified natural polymers, such as (hydroxypropyl)methylcellulose; basic amine-bearing polymers such as chitosan; acidic polymers obtainable from natural sources, such as alginic acid, hyaluronic acid, pectin, gum tragacanth, and karaya gum; and neutral synthetic polymers, such as polyvinyl alcohol or their mixtures. An additional group of mucoadhesive polymers includes natural and chemically modified cyclodextrin, especially hydroxypropyl-β-cyclodextrin. Such polymers may be present as free acids, bases, or salts, usually in a final concentration of about 0.01% to about 0.5% by weight.

A suitable bioadhesive macromolecule is the family of acrylic acid polymers and copolymers, (e.g., Carbopol®). These polymers contain the general structure —[CH2-CH(COOH)—]n. Hyaluronic acid and other biologically-derived polymers may be used.

Exemplary bioadhesive or mucoadhesive macromolecules have a molecular weight of at least 50 kDa, or at least 300 kDa, or at least 1,000 kDa. Favored polymeric ionizable macromolecules have not less than 2 mole percent acidic groups (e.g., COOH, SO3H) or basic groups (NH2, NRH, NR2), relative to the number of monomeric units. The acidic or basic groups can constitute at least 5 mole percent, or at least 10 mole percent, or at least 25, at least 50 more percent, or even up to 100 mole percent relative to the number of monomeric units of the macromolecule.

Yet, another group of mucoadhesive agent includes inorganic gelling agents such as silicon dioxide (fumed silica), including but not limited to, AEROSIL 200 (DEGUSSA).

Many mucoadhesive agents are known in the art to also possess gelling properties.

The foam composition may contain a film forming component. The film forming component may include at least one water-insoluble alkyl cellulose or hydroxyalkyl cellulose. Exemplary alkyl cellulose or hydroxyalkyl cellulose polymers include ethyl cellulose, propyl cellulose, butyl cellulose, cellulose acetate, hydroxypropyl cellulose, hydroxybutyl cellulose, and ethylhydroxyethyl cellulose, alone or in combination. In addition, a plasticizer or a cross linking agent may be used to modify the polymer's characteristics. For example, esters such as dibutyl or diethyl phthalate, amides such as diethyldiphenyl urea, vegetable oils, fatty acids and alcohols such as oleic and myristyl acid may be used in combination with the cellulose derivative.

In one or more embodiments, the composition of the present invention includes a phase change polymer, which alters the composition behavior from fluid-like prior to administration to solid-like upon contact with the target mucosal surface. Such phase change results from external stimuli, such as changes in temperature or pH and exposure to specific ions (e.g., Ca²⁺).

Non-limiting examples of phase change polymers include poly(N-isopropylamide), Poloxamer 407® and Smart-Gel® (Poloxamer+PAA).

The polymeric agent is present in an amount in the range of about 0.01% to about 5.0% by weight of the foam composition. In one or more embodiments, it is typically less than about 1 wt % of the foamable composition.

Surface-active agents (also termed “surfactants”) include any agent linking oil and water in the composition, in the form of emulsion. A surfactant's hydrophilic/lipophilic balance (HLB) describes the emulsifier's affinity toward water or oil. The HLB scale ranges from 1 (totally lipophilic) to 20 (totally hydrophilic), with 10 representing an equal balance of both characteristics. Lipophilic emulsifiers form water-in-oil (w/o) emulsions; hydrophilic surfactants form oil-in-water (o/w) emulsions. The HLB of a blend of two emulsifiers equals the weight fraction of emulsifier A times its HLB value plus the weight fraction of emulsifier B times its HLB value (weighted average).

Any surface-active agent or combinations thereof may be used as surface-active agent. According to one or more embodiments of the present invention, the surface-active agent has a hydrophilic lipophilic balance (HLB) between about 9 and about 14, which is the required HLB (the HLB required to stabilize an O/W emulsion of a given oil) of most oils and hydrophobic solvents. Thus, in one or more embodiments, the composition is a single surface active agent having an HLB value between about 9 and 14, and in one or more embodiments, the composition is more than one surface active agent and the weighted average of their HLB values is between about 9 and about 14.

The surface-active agent is selected from anionic, cationic, nonionic, zwitterionic, amphoteric and ampholytic surfactants, as well as mixtures of these surfactants. Such surfactants are well known to those skilled in the therapeutic and cosmetic formulation art. Nonlimiting examples of possible surfactants include polysorbates, such as polyoxyethylene (20) sorbitan monostearate (Tween 60) and poly(oxyethylene) (20) sorbitan monooleate (Tween 80); poly(oxyethylene) (POE) fatty acid esters, such as Myrj 45, Myrj 49, Myrj 52 and Myrj 59; poly(oxyethylene)alkylyl ethers, such as poly(oxyethylene) cetyl ether, poly(oxyethylene) palmityl ether, polyethylene oxide hexadecyl ether, polyethylene glycol cetyl ether, brij 38, brij 52, brij 56 and brij W1; sucrose esters, partial esters of sorbitol and its anhydrides, such as sorbitan monolaurate and sorbitan monolaurate; mono or diglycerides, isoceteth-20, sodium methyl cocoyl taurate, sodium methyl oleoyl taurate, sodium lauryl sulfate, triethanolamine lauryl sulfate and betaines.

In one or more embodiments of the present invention, the surface-active agent includes at least one non-ionic surfactant. Ionic surfactants are known to be irritants. Therefore, non-ionic surfactants are preferred in applications including sensitive tissue such as found in most mucosal tissues, especially when they are infected or inflamed. We have surprisingly found that non-ionic surfactants alone provide foams of excellent quality, i.e. a score of “E” according to the grading scale discussed herein below.

In one or more embodiments, the surface active agent includes a mixture of at least one non-ionic surfactant and at least one ionic surfactant in a ratio in the range of about 100:1 to 6:1. In one or more embodiments, the non-ionic to ionic surfactant ratio is greater than about 6:1, or greater than about 8:1; or greater than about 14:1, or greater than about 16:1, or greater than about 20:1.

In one or more embodiments of the present invention, a combination of a non-ionic surfactant and an ionic surfactant (such as sodium lauryl sulphate and cocamidopropylbetaiine) is employed, at a ratio of between 1:1 and 20:1, or at a ratio of 4:1 to 10:1. The resultant foam has a low specific gravity, e.g., less than 0.1 g/ml.

It has been surprisingly discovered that the solubilizing phenomenon, attributed to the kit of the present invention is especially pronounced when a combination of at least one non-ionic surfactant having HLB of less than 9 and at least one non-ionic surfactant having HLB of equal or more than 9 is employed. The ratio between the at least one non-ionic surfactant having HLB of less than 9 and the at least one non-ionic surfactant having HLB of equal or more than 9, is between 1:8 and 8:1, or at a ratio of 4:1 to 1:4. The resultant HLB of such a blend of at least two emulsifiers is between about 9 and about 14.

Thus, in an exemplary embodiment, a combination of at least one non-ionic surfactant having HLB of less than 9 and at least one non-ionic surfactant having HLB of equal or more than 9 is employed, at a ratio of between 1:8 and 8:1, or at a ratio of 4:1 to 1:4, wherein the HLB of the combination of emulsifiers is between about 9 and about 14.

In one or more embodiments of the present invention, the surface-active agent includes mono-, di- and tri-esters of sucrose with fatty acids (sucrose esters), prepared from sucrose and esters of fatty acids or by extraction from sucro-glycerides. Suitable sucrose esters include those having high monoester content, which have higher HLB values.

The total surface active agent is in the range of about 0.1 to about 5% of the foamable composition, and is typically less than about 2% or less than about 1%.

A foam adjuvant is optionally included in the foamable compositions of the present invention to increase the foaming capacity of surfactants and/or to stabilize the foam. In one or more embodiments of the present invention, the foam adjuvant agent includes fatty alcohols having 15 or more carbons in their carbon chain, such as cetyl alcohol and stearyl alcohol (or mixtures thereof). Other examples of fatty alcohols are arachidyl alcohol (C20), behenyl alcohol (C22), 1-triacontanol (C30), as well as alcohols with longer carbon chains (up to C50). Fatty alcohols, derived from beeswax and including a mixture of alcohols, a majority of which has at least 20 carbon atoms in their carbon chain, are especially well suited as foam adjuvant agents. The amount of the fatty alcohol required to support the foam system is inversely related to the length of its carbon chains.

In one or more embodiments of the present invention, the foam adjuvant agent includes fatty acids having 16 or more carbons in their carbon chain, such as hexadecanoic acid (C16) stearic acid (C18), arachidic acid (C20), behenic acid (C22), octacosanoic acid (C28), as well as fatty acids with longer carbon chains (up to C50), or mixtures thereof. As for fatty alcohols, the amount of fatty acids required to support the foam system is inversely related to the length of its carbon chain.

Optionally, the carbon atom chain of the fatty alcohol or the fatty acid may have at least one double bond. A further class of foam adjuvant agent includes a branched fatty alcohol or fatty acid. The carbon chain of the fatty acid or fatty alcohol also can be substituted with a hydroxyl group, such as 12-hydroxy stearic acid.

The foam adjuvant according to one or more embodiments of the present invention includes a mixture of fatty alcohols, fatty acids and hydroxy fatty acids and derivatives thereof in any proportion, providing that the total amount is 0.1% to 5% (w/w) of the carrier mass. More preferably, the total amount is 0.4%-2.5% (w/w) of the carrier mass.

While fatty alcohols and fatty acids serve to stabilize the resultant foam composition, they often provide additional therapeutic properties. Long chain saturated and mono unsaturated fatty alcohols, e.g., stearyl alcohol, erycyl alcohol, arachidyl alcohol and docosanol have been reported to possess antiviral, anti infective, antiproliferative and anti-inflammatory properties (U.S. Pat. No. 4,874,794). Longer chain fatty alcohols, e.g., tetracosanol, hexacosanol, heptacosanol, octacosanol, triacontanol, etc. are also known for their metabolism modifying properties and tissue energizing properties. Long chain fatty acids have also been reported to possess anti-infective characteristics. Thus, the therapeutic or cosmetic carrier, containing the foam adjuvant agent of the present invention provides an extra therapeutic benefit in comparison with currently used vehicles, which are inert and non-active.

The therapeutic foam of the present invention may further optionally include a variety of formulation excipients, which are added in order to fine-tune the consistency of the formulation, protect the formulation components from degradation and oxidation and modify their consistency. Such excipients may be selected, for example, from stabilizing agents, antioxidants, humectants, preservatives, colorant and odorant agents and other formulation components, used in the art of formulation.

Aerosol propellants are used to generate and administer the foamable composition as a foam. The total composition including propellant, foamable compositions and optional ingredients is referred to as the foamable carrier. The propellant makes up about 3% to about 25 wt % of the foamable carrier. Examples of suitable propellants include volatile hydrocarbons such as butane, propane, isobutane or mixtures thereof, and fluorocarbon gases.

By including an appropriate therapeutic azole and optional active agents in the compositions of the present invention, the composition are useful in treating a patient having any one of a variety of dermatological disorders (also termed “dermatoses”), such as classified in a non-limiting exemplary manner according to the following groups:

Dermatitis including Contact Dermatitis, Atopic Dermatitis, Seborrheic Dermatitis, Nummular Dermatitis, Chronic Dermatitis of the hands and feet, Generalized Exfoliative Dermatitis, Stasis Dermatitis; Lichen Simplex Chronicus; Diaper rash;

Bacterial Infections including Cellulitis, Acute Lymphangitis, Lymphadenitis, Erysipelas, Cutaneous Abscesses, Necrotizing Subcutaneous Infections, Staphylococcal Scalded Skin Syndrome, Folliculitis, Furuncles, Hidradenitis Suppurativa, Carbuncles, Paronychial Infections, Erythrasma;

Fungal Infections including Dermatophyte Infections, Yeast Infections; Parasitic Infections including Scabies, Pediculosis, Creeping Eruption;

Viral Infections;

Disorders of Hair Follicles and Sebaceous Glands including Acne, Rosacea, Perioral Dermatitis, Hypertrichosis (Hirsutism), Alopecia, including male pattern baldness, alopecia areata, alopecia universalis and alopecia totalis; Pseudofolliculitis Barbae, Keratinous Cyst;

Scaling Papular Diseases including Psoriasis, Pityriasis Rosea, Lichen Planus, Pityriasis Rubra Pilaris;

Benign Tumors including Moles, Dysplastic Nevi, Skin Tags, Lipomas, Angiomas, Pyogenic Granuloma, Seborrheic Keratoses, Dermatofibroma, Keratoacanthoma, Keloid;

Malignant Tumors including Basal Cell Carcinoma, Squamous Cell Carcinoma, Malignant Melanoma, Paget's Disease of the Nipples, Kaposi's Sarcoma;

Reactions to Sunlight including Sunburn, Chronic Effects of Sunlight, Photosensitivity;

Bullous Diseases including Pemphigus, Bullous Pemphigoid, Dermatitis Herpetiformis, Linear Immunoglobulin A Disease;

Pigmentation Disorders including Hypopigmentation such as Vitiligo, Albinism and Postinflammatory hypopigmentation and Hyperpigmentation such as Melasma (chloasma), Drug-induced hyperpigmentation, Postinflammatory hyperpigmentation;

Disorders of Cornification including Ichthyosis, Keratosis Pilaris, Calluses and Corns, Actinic keratosis;

Pressure Sores;

Disorders of Sweating; and

Inflammatory reactions including Drug Eruptions, Toxic Epidermal Necrolysis; Erythema Multiforme, Erythema Nodosum, Granuloma Annulare.

According to one or more embodiments of the present invention, the compositions are also useful in the therapy of non-dermatological disorders by providing transdermal delivery of an active azole that is effective against non-dermatological disorders.

The same advantage is expected when the composition is topically applied to mucosal membranes, the oral cavity, the vagina and the rectum to treat conditions such as chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, cnal abscess/fistula, anal cancer, anal fissure, anal warts, Crohn's disease, hemorrhoids, anal itch, pruritus ani, fecal incontinence, constipation, polyps of the colon and rectum.

In another aspect, the present invention provides a method of designing a therapeutic kit including, a therapeutic azole with increased solubility, which, in turn, provides a higher concentration of solubilized azole at the treatment site, comprising the steps of

(1) selecting a therapeutic azole;

(2) establishing a concentration higher than the expected solubility concentration of said therapeutic azole in the composition, as detected microscopically by as detected microscopically by counting more than 20 or more than 50 crystals in an area of 1 mm² at 100× magnification;

(3) screening a panel of valves, having varying numbers and sizes of apertures; and

(4) identifying a range of valves, having an optimal number of apertures and aperture sizes, wherein therapeutic azole in the foamed product is more soluble than the azole in the foamable composition prior its to release from the aerosol assembly. An increase in solubility of at least 0.1 wt % or at least 0.2 wt %.

The following examples exemplify the therapeutic kits and pharmacological compositions and methods described herein. The examples are for the purposes of illustration only and are not intended to be limiting of the invention.

### Example 1

### Miconazole Oil in Water Compositions

| | Composition No: | | | |
|---|---|---|---|---|
| | MN 1 | MN 2 | MN 3 | MN 4 |
| | % | | | |
| Ingredient | | | | |
| Miconazole nitrate | 0.8 | 1.2 | 0.40 | 0.80 |
| Water | 72.93 | 72.53 | 73.59 | 73.19 |
| Mineral oil | 5.60 | 5.60 | 5.60 | 5.60 |
| Isopropyl palmitate | 5.60 | 5.60 | 5.60 | 5.60 |
| Sorbitan stearate (Span 60) | 2.00 | 2.00 | 2.00 | 2.00 |
| PPG15-stearyl ether | 1.00 | 1.00 | 1.00 | 1.00 |
| Stearic acid | 0.85 | 0.85 | 0.85 | 0.85 |
| Glyceryl monostearate | 0.45 | 0.45 | 0.45 | 0.45 |
| Xanthan gum | 0.26 | 0.26 | 0.26 | 0.26 |
| Methocel K100M | 0.26 | 0.26 | — | — |
| Preservative | 0.25 | 0.25 | 0.25 | 0.25 |
| Propellant | 10.00 | 10.00 | 10.00 | 10.00 |
| Total | 100 | 100 | 100 | 100 |
| Composition Properties | | | | |
| Emulsion color | White | White | White | White |
| Crystals Observed in Emulsion | 50-100 | >100 | 50-100 | >100 |
| (per microscope screen, X100 | | | | |
| magnification) | | | | |
| Crystals Observed in Foam | 25 | 45 | 10 | 50 |
| (per microscope screen, X100 | | | | |
| magnification) | | | | |
| Foam Density | 0.06 | 0.06 | 0.08 | 0.08 |
| | Composition No: | | | |
| | MN 5 | MN 6 | MN 7 | MN 7 |
| | % | | | |
| Ingredient | | | | |
| Miconazole nitrate | 0.4 | 1.2 | 0.40 | 0.80 |
| Water | 74.54 | 73.74 | 74.82 | 74.02 |
| Mineral oil | 5.60 | 5.60 | 5.60 | 5.60 |
| Capric caprylic triglyceride | 5.60 | 5.60 | 5.60 | 5.60 |
| PEG-40 stearate | 2.80 | 2.80 | 2.80 | 2.80 |
| Polysorbate 80 | 0.90 | 0.90 | 0.90 | 0.90 |
| Stearic acid | 0.90 | 0.90 | 0.90 | 0.90 |
| Glyceryl monostearate | 0.45 | 0.45 | 0.45 | 0.45 |
| Xanthan gum | 0.28 | 0.28 | 0.28 | 0.28 |
| Methocel K100M | 0.28 | 0.28 | — | — |
| Preservative | 0.25 | 0.25 | 0.25 | 0.25 |
| Propellant | 8.00 | 8.00 | 8.00 | 8.00 |
| Total | 100 | 1001 | 100 | 100 |
| Composition Properties | | | | |
| Emulsion color | White | White | White | White |
| Crystals Observed in Emulsion | 50-100 | >100 | >100 | >100 |
| (per microscope screen, X100 | | | | |
| magnification) | | | | |
| Crystals Observed in Foam | 6 | 36 | 25 | 25 |
| (per microscope screen, X100 | | | | |
| magnification) | | | | |
| Foam Density | 0.06 | 0.06 | 0.08 | 0.08 |

### Example 2

### Metronidazole Oil in Water Foamable Compositions

| | Composition No: | | | | |
|---|---|---|---|---|---|
| | MZ 1 | MZ 2 | MZ 3 | MZ 4 | MZ-5 |
| | % | | | | |
| Ingredient | | | | | |
| Metronidazole | 1.0 | 1.0 | 1.4 | 1.0 | 1.4 |
| Water | 73.94 | 71.94 | 73.54 | 74.22 | 73.82 |
| Mineral oil | 5.60 | 5.60 | 5.60 | 5.60 | 5.60 |
| Capric caprylic | 5.60 | 5.60 | 5.60 | 5.60 | 5.60 |
| triglyceride | | | | | |
| PEG-40 stearate | 2.80 | 2.80 | 2.80 | 2.80 | 2.80 |
| Propylene glycol | — | 2.00 | — | — | — |
| Polysorbate 80 | 0.90 | 0.90 | 0.90 | 0.90 | 0.90 |
| Stearic acid | 0.90 | 0.90 | 0.90 | 0.90 | 0.90 |
| Glyceryl monostearate | 0.45 | 0.45 | 0.45 | 0.45 | 0.45 |
| Xanthan gum | 0.28 | 0.28 | 0.28 | 0.28 | 0.28 |
| Methocel K100M | 0.28 | 0.28 | 0.28 | — | — |
| Preservative | 0.25 | 0.25 | 0.25 | 0.25 | 0.25 |
| Propellant | 8.00 | 8.00 | 8.00 | 8.00 | 8.00 |
| Total | 11.06 | 100 | 11.06 | 100 | 100 |
| Composition Properties | | | | | |
| Emulsion color | White | White | White | White | White |
| Crystals Observed | 33 | 55 | >100 | 30 | >100 |
| in Emulsion (per | | | | | |
| microscope screen, | | | | | |
| X100 magnification) | | | | | |
| Crystals Observed | 2 | 3 | 5 | 6 | 10 |
| in Foam (per | | | | | |
| microscope screen, | | | | | |
| X100 magnification) | | | | | |

### Example 3

### Skin Penetration Studies, Demonstrating Enhanced Skin Penetration of Metronidazole, Using the Kit of the Present Invention

### Aim:

The aim of this study was to compare the dermal and transdermal penetration of Metronidazole formulated at 1% in Compositions No. MZ 1 and MZ 2, as provided in Example 2, in comparison with a commercial 1% Metronidazole cream, namely “Noritate” cream (Dermik).

### Materials and Methods:

The study was conducted using excised human skin mounted in a flow-through diffusion cell over a 16-hour period. Three skin samples from three women were used. A target amount of 10 mg of each formulation (100 μg of Metronidazole) was applied to a skin surface of 1 cm². Concentrations of Metronidazole in the receptor fluid fractions over time and the remaining Metronidazole in the skin at the end of the study were assayed by HPLC.

### Results:

The following table summarizes the amounts of Metronidazole in the epidermis (E) and dermis (D), as well as the amount of Metronidazole that was absorbed transdermally (T).

| | MZ 1 | MZ 2 | Noritate |
|---|---|---|---|
| Applied amount (μg) | 94 | 87.6 | 96.8 |
| Epidermis + Stratum Corneum | 4.87 | 4.26 | 1.99 |
| (μg) | | | |
| % of the Applied Dose | 5.18% | 4.86% | 2.06% |
| Dermis (μg) | 1.72 | 1.87 | 0.65 |
| % of the Applied Dose | 1.83% | 2.13% | 0.67% |
| Total Skin (E + D) (μg) | 6.59 | 6.13 | 2.64 |
| % of the Applied Dose | 7.01% | 7.00% | 2.73% |
| Transdermal Absorption (μg) | 3.54 | 5.85 | 2.04 |
| % of the Applied Dose | 3.77% | 6.68% | 2.11% |
| Total Penetrated Amount | 10.13 | 11.98 | 4.68 |
| (E + D + T) (μg) | | | |
| % of the Applied Dose | 10.78% | 13.68% | 4.83% |

The following observations were made from these results:

1. The dermal penetration of Metronidazole from both foam compositions was about 2.5 times better than the corresponding penetration from Noritate. This enhanced dermal drug residence is expected to improve the activity of the drug in its target site—the skin.

2. The drug released from both foams was found both in the epidermis and dermis, thus ensuring its biological activity in all skin layers.

3. Transdermal delivery was enhanced by propylene glycol.

Thus, it can be concluded that the enhanced solubility, as provided by the kit of the present invention is useful in enhancing the effectiveness of a therapeutic azole.

### Example 4

### Additional Metronidazole Oil in Water Compositions (30% Hydrophobic Carrier)

| | MZ 3 | MZ 4 | MZ 5 | MZ 6 | MZ 7 | MZ 8 |
|---|---|---|---|---|---|---|
| Metronidazole | 1.2% | 1.4% | 1.6% | 1.2% | 1.4% | 1.6% |
| MCT | 30.0% | 30.0% | 30.0% | 30.0% | 30.0% | 30.0% |
| PEG-40 | 3.0% | 3.0% | 3.0% | 3.0% | 3.0% | 3.0% |
| Stearate | | | | | | |
| Stearyl alcohol | 1.0% | 1.0% | 1.0% | 1.0% | 1.0% | 1.0% |
| Polysorbate 80 | 1.0% | 1.0% | 1.0% | 1.0% | 1.0% | 1.0% |
| Glyceryl | 0.5% | 0.5% | 0.5% | 0.5% | 0.5% | 0.5% |
| Sterarte | | | | | | |
| Cocamido- | 0.5% | 0.5% | 0.5% | 0.5% | 0.5% | 0.5% |
| betaine | | | | | | |
| Xanthan gum | 0.3% | 0.3% | 0.3% | 0.3% | 0.3% | 0.3% |
| Methyl- | 0.3% | 0.3% | 0.3% | 0.3% | 0.3% | 0.3% |
| cellulose | | | | | | |
| Phenonip | 0.3% | 0.3% | 0.3% | 0.3% | 0.3% | 0.3% |
| Butane/Propane | — | — | — | 16.0% | 16.0% | 16.0% |
| 80/20 | | | | | | |
| Purified water | To 100 | To 100 | To 100 | To 100 | To 100 | To 100 |

Composition Properties

| | | | | | | |
|---|---|---|---|---|---|---|
| Crystals | 5-25 | 10-25 | 15-35 | | | |
| Observed in | | | | | | |
| Emulsion | | | | | | |
| (per 1 mm²) | | | | | | |
| Crystals | | | | 2-4 | 3-6 | 5-9 |
| Observed in | | | | | | |
| Foam (per | | | | | | |
| 1 mm²) | | | | | | |

### Example 5

### Microscopic Comparison of Crystals in 1% Metronidazole Compositions of the Present Invention and the Commercial 1% Metronidazole Topical Product—Noritate (Dermik Laboratories Ltd.)

Samples of (1) 1% Metronidazole compositions emulsion; (2) 1% Metronidazole composition foam; and (3) 1% Metronidazole topical product—Noritate (Dermik) were examined microscopically at ×100 magnification. Typical microscopic pictures are provided below. Notably, the skin penetration results, as described in Example 3 hereinabove, corroborate with the high solubility of Metronidazole in the compositions of the present invention.

### Example 6

### Ketoconazole Oil in Water Compositions

| | Composition Code: | | | | | |
|---|---|---|---|---|---|---|
| | KF 1 | KF 2 | KF 3 | KF 4 | KF 5 | KF 6 |
| Ingredient | % | | | | | |
| Ketoconazole | 0.01 | 0.10 | 0.20 | 0.30 | 0.50 | 1.00 |
| Water | 72.82 | 71.83 | 71.73 | 72.53 | 72.33 | 71.83 |
| Mineral oil | 14.10 | 15.00 | 15.00 | 14.10 | 14.10 | 14.10 |
| Span 60 | 2.00 | 2.00 | 2.00 | 2.00 | 2.00 | 2.00 |
| PPG15- | 1.00 | 1.00 | 1.00 | 1.00 | 1.00 | 1.00 |
| stearyl ether | | | | | | |
| Stearic acid | 0.85 | 0.85 | 0.85 | 0.85 | 0.85 | 0.85 |
| Glyceryl | 0.45 | 0.45 | 0.45 | 0.45 | 0.45 | 0.45 |
| monostearate | | | | | | |
| Xanthan gum | 0.26 | 0.26 | 0.26 | 0.26 | 0.26 | 0.26 |
| Methocel | 0.26 | 0.26 | 0.26 | 0.26 | 0.26 | 0.26 |
| K100M | | | | | | |
| Phenochem | 0.25 | 0.25 | 0.25 | 0.25 | 0.25 | 0.25 |
| Propellant | 8.00 | 8.00 | 8.00 | 8.00 | 8.00 | 8.00 |
| Total | 100 | 100 | 100 | 100 | 100 | 100 |

Composition Properties (Foam vs. Emulsion)

| | | | | | | |
|---|---|---|---|---|---|---|
| Emulsion color | White | White | White | White | White | White |
| Crystals | None | Present | Present | Present | Present | Present |
| Observed in | | | | | | |
| Emulsion | | | | | | |
| Crystals | None | None | None | None | Present | Present |
| Observed in | | | | | | |
| Foam | | | | | | |
| Foam Density | 0.06 | 0.23 | 0.33 | 0.12 | 0.16 | 0.16 |

1. (canceled)

2. An aerosol container comprising a pressurized product and an outlet capable of releasing the pressurized product as a foam;
wherein said pressurized product comprises a foamable composition comprising:
i. a therapeutic azole, wherein the solubility of the azole in the composition before foaming is less than the solubility of the azole in the composition after foaming;
ii. at least one organic carrier selected from the group consisting of a hydrophobic organic carrier, a co-solvent, an emollient and mixtures thereof, at a concentration of about 2% to about 50% by weight;
iii. a surface-active agent;
iv. about 0.01% to about 5% by weight of at least one polymeric additive selected from the group consisting of a bioadhesive agent, a gelling agent, a film forming agent and a phase change agent;
v. water; and
vi. liquefied or compressed gas propellant at a concentration of about 3% to about 25% by weight of the total composition.

3. The container of claim 2, wherein the outlet comprises a valve.

4. The container of claim 3, wherein the valve comprises a stem with at least 1 aperture formed in said stem.

5. The container of claim 4, wherein the valve comprises a stem with 1 to 4 apertures formed in said stem.

6. The container of claim 4, wherein each said aperture formed in said stem has a diameter of about 0.2 mm to about 1 mm.

7. The container of claim 4, wherein each said aperture formed in said stem has a diameter of about 0.3 mm to about 0.8 mm.

8. The container of claim 4, wherein the sum of areas of all apertures in said stem is between about 0.01 mm2 and 1 mm2.

9. The container of claim 4, wherein the sum of areas of all apertures in said stem is between about 0.04 mm2 and 0.5 mm2.
